(19) United States
(12) Patent Application Publication
(10) Pub. No.: US 2001/0049541 A1
(43) Pub. Date: Dec. 6, 2001

(54) IMPLANTABLE HEART STIMULATION APPARATUS

(75) Inventors: GIANNI PLICCHI, BOLOGNA (IT); BRUNO GARBEROGLIO, TORINO (IT); GUIDO GAGGINI, MILANO (IT); EMANUELA MARCELLI, MACERATA (IT)

Correspondence Address: TERRY L WILES, POPOVICH & WILES PA, IDS CENTER, 80 SOUTH 8TH STREET SUITE 1902, MINNEAPOLIS, MN, 554022111

(73) Assignee: Sorin Biomedica Cardio S.p.A.

(21) Appl. No.: 09/299,862

(22) Filed: Apr. 26, 1999

(30) Foreign Application Priority Data
Jan. 28, 1999 (EP) 99830033.9

(51) Int. Cl. A61N 1/362

(52) U.S. Cl. 607/9

(57) ABSTRACT

An implantable heart-stimulation apparatus having a plurality of stimulation electrodes which can be applied to the heart muscle in one or more chambers of the heart in order to apply electrical stimulation signals thereto, and stimulation-control means for applying stimulation signals to the electrodes in accordance with a time plan determined individually and separately for each of the plurality of electrodes.

The preferred application is for the correction of myocardial electrical conduction defects, for example, in atrial fibrillation and cardiac insufficiency.

### FIELD OF THE INVENTION

The present invention relates to heart-stimulation techniques. In particular, this invention relates to improving the electro-mechanical characteristic of the function of the atria and the ventricles and/or in preventing arrhythmic events such as fibrillation also resulting from pathological anomalies of the conduction rate and of the duration of the refractory periods.

### BACKGROUND OF THE INVENTION

Human atrial fibrillation probably involves at least a limited establishment of interlaced re-entrant wavelets or the establishment of ectopic rapid-discharge foci, both of which phenomena can be treated by radio-frequency ablation. The wavelength of the reentry circuit is related to the rate of conduction and to the refractory period. Both of these parameters in turn depend on any drugs administered, on the heart rate and on atrial myotasis, on autonomic influences, and/or on the anisotropy of the myocardium. The functional circuit which can induce atrial stimulation within the atrium is therefore not stable in space or in time; it can be altered by electrical stimulation so as to prevent or to render more difficult the possible production of arrhythmia. In this connection, the use of atrial stimulation techniques in several sites has already been proposed. In particular, at least two different techniques have been proposed, as proved, for example, by the following works:

“Biatrial pacing and AF prevention” by J. C. Daubert, G. R. d'Allones, and P. Mabo in Working Group on Arrhythmias of the European Society of Cardiology; International meeting; Atrial fibrillation, Bologna, Italy—Oct. 12, 13, 14 1997, and “Prevention of recurrent atrial fibrillation with chronic dual site right atrial pacing” by S. Saksena, A. Prakash, M. Hill et al. in J. Am. Coll. Cardiol. 1996, 28:687-694.

In the technique described by Daubert et al, the leads are situated in the upper right atrium as well as in the central or distal portion of the coronary sinus in order to perform a selective stimulation and sensing action in the left atrium whereas, in the technique described by Saksena et al, the top electrode of the second lead is fixed to the edge of the ostium of the coronary sinus in a rear and lower position in the right atrium. In the technique of Daubert et al, the two atrial leads are connected to the atrial connector or port of a bipolar DDD pacemaker by means of a Y-shaped adapter, whereas in the technique of Saksena et al, the device is a normal DDD pacemaker and simultaneous stimulation in the two atrial sites is possible only in stimulated atrial cycles.

Another pathological situation which frequently arises in cardiac insufficiency, particularly where there is dilation and obstruction, consists of asynchronism of the contraction of the ventricular chambers caused precisely by an altered rate of conduction towards the two ventricles of the stimuli generated physiologically by stimulation. The asynchronism of these contractions produces an actual antagonistic effect between the deformations of the muscle masses of the two ventricles, which is revealed by a considerable reduction in heart performance or by actual muscular obstructions to the left-ventricle ejection stage.

At present the sole, incomplete approach to this problem consists of so-called simultaneous biventricular stimulation in which the stimulus is sent simultaneously to both of the two heart chambers in question by the positioning of two electrodes therein.

### SUMMARY OF THE INVENTION

The object of the present invention is to overcome the intrinsic limitations of the solutions described above.

In one aspect, this invention is an implantable heart-stimulation apparatus comprising a plurality of stimulation electrodes applied to the heart muscle in order to apply electrical stimulation signals thereto, and stimulation-control means adapted to apply stimulation signals to the electrodes in accordance with a time plan determined individually and separately for each electrode. The stimulation-control means generate the stimulation signals based on at least one parameter selected from the group of a time analysis of the electro-physiological signals recorded on the electrodes, an electro-stimulation stage in at least one of the electrodes, an estimate of the spatial positioning of the electrodes, heart functionality parameters detected externally or by implanted sensors, and aorto-ventricular pressure gradients.

The stimulation-control means is capable of activating a given stimulation sequence synchronously with an electrogram detected by one of the electrodes. One of the electrodes may be located in a position closest to the node of the atrial sinus. The stimulation sequence may be an atrial stimulation sequence. Preferably, electrodes are located in different heart chambers, and the stimulation-control means can apply stimulation signals at different times to electrodes in different chambers, the times being determined by optimization of hemodynamic parameters derived externally or by means of implantable sensors. Further, the stimulation-control means can apply the stimulation signals to the electrodes in a sequence independent of the location of the electrodes. The apparatus may include sensing means for transferring to the stimulation-control means sensing signals derived from the electrodes. The sensing means can communicate electro-physiological sensing signals derived separately from each electrode, in accordance with a programmable time plan. Electrodes can be implanted separately and independently of the other electrodes, or they may be disposed on a lead for simultaneous implantation.

In another aspect, this invention is an implantable heart-stimulation apparatus comprising a plurality of stimulation electrodes that can be applied to the heart muscle in order to apply electrical stimulation signals thereto, and a control unit in electrical communication with the plurality of stimulation electrodes and adapted to apply stimulation signals to the plurality of electrodes in a desired sequence.

In yet another aspect, this invention is a method of stimulating the heart by implanting a plurality of electrodes in desired locations in the heart; and applying stimulation signals to the electrodes according to a time sequence determined individually and separately for each electrode. The step of implanting electrodes may comprise implanting the electrodes in different heart chambers.

### BRIEF DESCRIPTION OF THE DRAWING

FIG. 1 shows a block diagram of the circuit structure of a multi-pole stimulator according to the invention.

### DETAILED DESCRIPTION OF THE PREFERRED EMBODIMENTS

The heart-stimulation apparatus according to the present invention comprises electrodes that are inserted permanently in the heart chambers. These electrodes can electrically stimulate the myocardium and detect the electro-physiological signal individually and locally.

The determination of the number of electrodes or poles, as well as the number of leads on which they are arranged, and also the selection of the anatomical sites in which to locate them, depend on the patient's specific arrhythmic condition. This is because the primary object of the device is to regularize the electro-physiological conduction processes in the individual heart chamber and/or between the various heart chambers, which object cannot be achieved by the known solutions which comprise a simultaneous stimulation of different regions.

For this purpose, the stimulator can be programmed so as to select, for example, suitable time sequences of stimuli which can be delivered by the various poles, and to receive electro-physiological signals in accordance with a time plan programmable individually for each pole, with the particular object of determining time sequences of progressive activation within a heart chamber or between several heart chambers, so as to reproduce or alter the natural rate of propagation of the spontaneous excitation.

The object of this type of stimulation is to improve the electro-mechanical characteristic of the function of the heart chambers (preventing, for example, competition between different chambers) and/or to prevent arrhythmic events, such as fibrillation also resulting from pathological anomalies of the conduction rate and of the duration of the refractory periods.

In a particularly preferred embodiment, the stimulator according to the invention is of the dual-chamber type in which the atrial stimulation and sensing lead is a multi-pole lead which can deliver pulses in accordance with a time plan programmable individually for each pole in order to bring about a progressive activity of the chamber such as to simulate normal electro-physiological conduction and to correct the anomalies of the rate of conduction of the local refractory periods of the myocardium which give rise to supraventricular arrhythmias.

Another particularly preferred embodiment consists of the execution of a selective stimulation of the right ventricle and of the left ventricle with a capability for suitable programming of the sequence and of the time interval both of the stimulation of the two ventricles and of the atrio-ventricular delays. The duration of the sequence of stimulation pulses, for example, atrial stimulation pulses (to prevent fibrillation) is programmable and can be deduced by time analysis of the electro-physiological signals recorded by the stimulator at the poles during the stages in which arrhythmia is absent, or by a single-pole electro-stimulation stage, or can even be estimated from the spatial positioning of the poles.

In the case of multi-site biventricular stimulation, the intervals of the stimulation sequence can be optimized by examination of the mechanical heart function deduced by external methods (e.g. by echocardiogram) or by special sensors included in the implantable device, for example, of the type described in U.S. Pat. No. 5,304,208 (Inguaggiato et al.), U.S. Pat. No. 5,496,351 (Plicchi et al.), or U.S. Pat. No. 5,609,612 (Plicchi et al.).

The atrial stimulation sequence may be activated in synchronism with the atrial electrogram detected by the electrode or pole nearest to the node of the atrial sinus. Alternatively, the stimulation control means may activate a given stimulation sequence automatically and asynchronously depending on the stimulator stand-by programming. The stimulation control means may also have a rate-responsive function.

The sequence of activation of the poles is programmable and does not necessarily follow their geometric progression for the purpose of a possible correction of the electro-physiological path of the atrial activation wavelets in the prevention of supraventricular arrhythmia. The activation of each individual pole involves the determination of a programmable blanking interval in all of the remaining poles.

In the case of an atrial stimulation sequence synchronous with the spontaneous activity, the regulation of the duration of the atrio-ventricular delay (the AV delay) has the atrial electrogram as a reference, whereas, in the case of an asynchronous sequence, the reference is the last atrial stimulus.

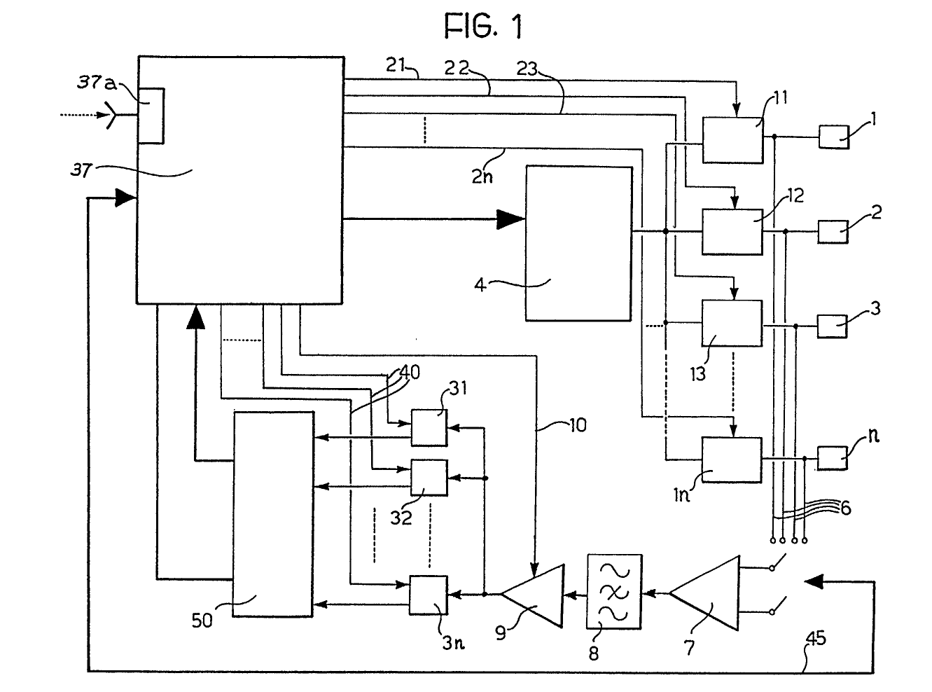

The invention will now be described, purely by way of non-limiting example, with reference to FIG. 1. A plurality of electrodes (or “poles”) that fits in sites in the heart suitable for bringing about a stimulation effect on the heart muscle by the application of electrical signals is indicated 1, 2, 3, . . . n. The structural characteristics of these electrodes, for example, the selection of constituent materials, any surface treatment, etc., are those currently used in known implantable defibrillators.

In particular, the term “electrode” or “pole” as used in the present description is intended to define any electrically-conductive member which can be associated with the heart muscle in a relationship suitable for the transmission of an electrical signal. Consequently, the electrodes or poles in question may either be configured as physically separate members which are thus intended to be fitted in distinct and separate respective myocardial sites. That is, each electrode may be implanted separately and independently of other electrodes. Alternatively, the electrodes may be associated in groups or sets with a supporting structure (“lead”) so as to be fitted in myocardial sites separately (in the sense that each electrode or pole can transfer—and detect—a respective signal to—and from—the myocardium) but not independently, since the electrodes or poles of each group disposed on a lead are implanted simultaneously (i.e., the entire lead is implanted).

With regard to the selection of the number n, the solution according to the invention can be implemented either in a minimal configuration comprising only two electrodes, or in more complex configurations with a number n equal, for example, to four, eight or ten (the selection of an even number is not, however, essential). The electrodes 1 to n may either be identical to one another or may have different shapes according to the sites in which they are to be positioned.

Power stage 4 of the implantable stimulator causes the voltage pulses to be applied to the electrodes 1 to n via respective electronic enabling switches 11, 12, 13, . . . , 1n. Switches or control gates 11, 12, 13, . . . , 1n are connected to respective enabling lines 21, 22, 23, . . . , 2n (thus allowing the signals generated in power stage 4 to pass selectively towards the electrodes 1, 2, . . . n). Switches or control gates 11 to 1n communicate with and are controlled by timing and control unit 37, preferably having an associated telemetering interface 37a of generally known type.

Control unit 37 electronically communicates with the electronic enabling switches, and thus the electrodes, by means of the lines 21, 22, 23, . . . 2n. Control unit 37 permits the characteristics of the stimulation signals applied to the myocardium to be programmed, particularly with regard to duration, amplitude, and polarity and to the ability to activate the pulses at the various electrodes 1, 2, 3, . . . , n sequentially in time (in accordance with a typical time-sharing scheme).

As a result, the above-described multi-pole configuration permits, in particular, the creation of an electrical stimulation field with variable geometry designed for the patient's anatomy-with a high degree of flexibility-by virtue of the ability of control unit 37 to program the emission of the control signals.

The stimulation may advantageously be activated by a sensing operation performed by the electrodes 1, 2, 3, . . . n. Sensing lines 6 are provided for this purpose and enable the electrical signals indicative of the local myocardial activity at the respective implantation site to be detected by the respective electrode so as to supply to control unit 37 a set of signals which enables the unit to see and to identify the occurrence of time anomalies in the myocardial electrical conduction.

The diagram of the appended drawing shows multiplexer 7 that enables the signal coming from a respective electrode 1, 2, 3, . . . n, to be detected selectively by one of lines 6. After anti-noise filtering performed in filter 8 and automatic gain control performed in automatic gain-control circuit (AGC) 9 controlled by control unit 37 by means of line 10, the sensing signals thus detected reach respective sampling circuits 31, 32, . . . , 3n, preferably of the sample-and-hold type, which are controlled by control unit 37 by means of enabling lines (generally indicted 40) in a manner coordinated with the driving of multiplexer 7 performed by control unit 37 by means of line 45.

The signals stored in circuits 31, 32, . . . 3n at any particular time (each relating to the sensing operation performed at the time in question by a respective electrode 1, 2, . . . n) are sent to analog/digital conversion circuit 50 for transmission to control unit 37. The application of a stimulation signal to one of the electrodes (in) may result in a refractory state (blanking) of the sensing means. Control unit 37 can thus carry out a time analysis of the electro-physiological signals recorded in electrodes 1, 2, . . . n during stages in which arrhythmia is absent or after a single-pole electro-stimulation stage (possibly taking account of the spatial positioning of the poles) so as to be able to deliver the stimulation pulses in accordance with a time plan programmable individually for each electrode. This permits, for example, progressive activation of the chamber treated such as to simulate the normal electro-physiological conduction and to correct anomalies of the rate of conduction of the local refractory periods of the myocardium such as to give rise to supraventricular arrhythmias. Naturally, this relates to the prevention of atrial fibrillation situations.

In the case of cardiac insufficiency, on the other hand, the regulation of the activation times of poles 1, 2, . . . n is controlled by control unit 37 either in response to the implanted heart-function signal or on the basis of external hemodynamic parameters such as the cardiac output or the aorto-ventricular pressure gradient.

Clearly, the various electronic components shown in the drawing may be in the form of discrete blocks or components, or may be integrated in a single circuit. Naturally, the principle of the invention remaining the same, the details of construction and forms of embodiment may be varied widely with respect to those described and illustrated, without thereby departing from the scope of the present invention as defined by the following claims.

What is claimed is:

1. An implantable heart-stimulation apparatus comprising:
a plurality of stimulation electrodes applied to the heart muscle in order to apply electrical stimulation signals thereto; and
stimulation-control means adapted to apply stimulation signals to the electrodes in accordance with a time plan determined individually and separately for each electrode.

2. The apparatus according to claim 1, wherein the stimulation-control means generate the stimulation signals based on at least one parameter selected from the group of:
a time analysis of the electro-physiological signals recorded on the electrodes, an electro-stimulation stage in at least one of the electrodes, an estimate of the spatial positioning of the electrodes, heart functionality parameters detected externally or by implanted sensors, and aorto-ventricular pressure gradients.

3. The apparatus according to claim 1, wherein the stimulation-control means activate a given stimulation sequence synchronously with an electrogram detected by one of the electrodes.

4. The apparatus according to claim 3, wherein the one of the electrodes is located in a position closest to the node of the atrial sinus, and wherein the stimulation sequence is an atrial stimulation sequence.

5. The apparatus according to claim 1, wherein the plurality of electrodes is associated with different heart chambers, and wherein the stimulation-control means applies stimulation signals at different times to electrodes in different chambers, the times being determined by optimization of hemodynamic parameters derived externally or by means of implantable sensors.

6. The apparatus according to claim 1, wherein the stimulation-control means apply the stimulation signals to the electrodes in a sequence independent of the location of the electrodes.

7. The apparatus according to claim 1, further comprising sensing means for transferring to the stimulation-control means sensing signals derived from the electrodes.

8. The apparatus according to claim 1, further comprising sensing means for transferring to the stimulation-control means respective electro-physiological sensing signals derived separately from each electrode of the plurality of electrodes, in accordance with a programmable time plan.

9. The apparatus according to claim 1 wherein at least some of the electrodes can be implanted separately and independently of the other electrodes.

10. The apparatus according claim 1, wherein at least some of the electrodes are disposed on a lead for simultaneous implantation.

11. An implantable heart-stimulation apparatus comprising:
a plurality of stimulation electrodes that can be applied to the heart muscle in order to apply electrical stimulation signals thereto; and
a control unit in electrical communication with the plurality of stimulation electrodes and adapted to apply stimulation signals to the plurality of electrodes in a desired sequence.

12. The apparatus according to claim 11, wherein the control unit generates the stimulation signals based on at least one parameter selected from the group of: a time analysis of the electro-physiological signals recorded on the electrodes, an electro-stimulation stage in at least one of the electrodes, an estimate of the spatial positioning of the electrodes, heart functionality parameters detected externally or by implanted sensors, and aorto-ventricular pressure gradients.

13. The apparatus according to claim 11, wherein the control unit activates a given stimulation sequence synchronously with an electrogram detected by one of the electrodes.

14. The apparatus according to claim 13, wherein one of the electrodes is located in a position closest to the node of the atrial sinus, and wherein the stimulation sequence is an atrial stimulation sequence.

15. The apparatus according to claim 11, wherein the plurality of electrodes is associated with different heart chambers, and wherein the control unit applies stimulation signals at different times to electrodes in different chambers, the times being determined by optimization of hemodynamic parameters derived externally or by means of implantable sensors.

16. The apparatus according to claim 11, wherein the control unit applies the stimulation signals to the electrodes in a sequence independent of the location of the electrodes.

17. The apparatus according to claim 11, further comprising a sensor for transferring to the stimulation-control means sensing signals derived from the electrodes.

18. The apparatus according to claim 11, further comprising a sensor for transferring to the stimulation-control means respective electro-physiological sensing signals derived separately from each electrode, in accordance with a programmable time plan.

19. A method of stimulating the heart comprising:
implanting a plurality of electrodes in desired locations in the heart; and
applying stimulation signals to the electrodes according to a time sequence determined individually and separately for each electrode.

20. The method according to claim 19, wherein the step of applying stimulation signals comprises applying the signals at times determined by optimization of externally derived hemodynamic parameters or by means of implantable sensors.

21. The method according to claim 19, wherein the step of implanting electrodes comprises implanting the electrodes in different heart chambers.
